(19) United States
(12) Patent Application Publication
(10) Pub. No.: US 2009/0018633 A1
(43) Pub. Date: Jan. 15, 2009

(54) PROTECTOR FOR AN INSERTABLE OR IMPLANTABLE MEDICAL DEVICE

(75) Inventors: Jeffrey S. Lindquist, Maple Grove, MN (US); Peter Edelman, Maple Grove, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 11/775,324

(22) Filed: Jul. 10, 2007

(51) Int. Cl. A61B 19/02; A61F 2/06; A61M 25/10

(52) U.S. Cl. 623/1.11; 206/204; 206/438; 604/103.02

(57) ABSTRACT

A protector for a medical device including a tubular member, the tubular member including a polymer matrix material and a scavenger for moisture, oxygen or a combination thereof dispersed in the polymer matrix material.

### FIELD OF THE INVENTION

The present invention relates to the field of catheter assemblies used for the delivery of medical devices, in particular medical device delivery systems having a medical device protector means.

### BACKGROUND OF THE INVENTION

A stent is an implantable medical device introduced into a body lumen and is well known in the art. Typically, a stent is implanted in a blood vessel at the site of a stenosis or aneurysm endoluminally, i.e. by so-called “minimally invasive techniques” in which the stent, in a radially reduced configuration, optionally restrained in a radially compressed configuration by a sheath and/or catheter, is delivered by a stent delivery system or “introducer” to the site where it is required. The introducer can enter the body from an access location outside the body, such as through the patient's skin, or by a “cut down” technique in which the entry vessel is exposed by minor surgical means.

Stents are often inserted during percutaneous transluminal coronary angioplasty or PTCA procedures to reduce the risk of restenosis.

A further means of reducing the risk of restenosis is to treat the stent with a therapeutic agent that assists in preventing restenosis. One method is to bioengineer coatings that release the therapeutic agent in a controlled manner, either from the coating itself, or from the stent. Of course, therapeutic agents may be added for other reasons as well such as to decrease inflammation or to have an antiobiotic effect, for example.

Some of these coatings are susceptible to environmental exposure such as environmental moisture, oxygen and light, for example. For example, coatings, also referred to in the art as excipients, that may include any of a variety of bioerodable or biodegradable polymers such as those comprising l-lactide or d,l-lactide, with or without glycolide, can be hydrolytically unstable when subjected to atmospheric moisture. Some therapeutic agents are also hydrolytically unstable, for example, Everolimus. A variety of packaging methods have been developed for just these reasons. For example, see U.S. Pat. No. 7,000,770 and U.S. Patent Publication Nos. 2005/0268573 and 2006/0260967.

Stent protectors are used to protect the stent and stent coatings before the stent and catheter assembly are introduced and subsequently the stent deployed and implanted into a body lumen. The stent protector protects the stent from physical damage or contamination due to the transfer of unwanted material and is removed at the time of use to permit delivery and deployment of the stent in the body of a patient. Examples of stent protectors are provided in commonly assigned U.S. Pat. Nos. 6,991,639, 6,783,542, 6,764,504, 6,416,529, 6,152,944, 5,893,868, and 5,342,307, each of which is incorporated by reference herein in its entirety.

It would be desirable to have an improved stent protector which reduces the exposure of the stent to environmental moisture, oxygen and light.

### SUMMARY OF THE INVENTION

The present invention relates to an improved protector for a medical device including at least one scavenger for moisture, oxygen, ultraviolet (UV) radiation, or a combination thereof.

In specific embodiments, the protector is disposed about a stent, an expandable medical balloon or both, and the stent, the medical balloon or both may be disposed about the distal end of a catheter assembly.

In one aspect, the protector includes a tubular member formed from at least one layer, wherein the at least one layer is formed from a polymer composition including a polymer matrix material and at least one scavenger for moisture, oxygen, or a combination thereof dispersed in the polymer matrix material.

In another aspect, the protector is a multilayer protector including at least two layers wherein one layer is formed from a polymer composition including a polymer matrix material and at least one scavenger for moisture, oxygen, ultraviolet (UV) radiation or a combination thereof dispersed in the polymer matrix material.

In another aspect, the present invention relates to a method of making the protector according to the invention including blending at least one polymer matrix material with at least one scavenger for moisture, oxygen or combination thereof, and forming a tubular member.

Alternatively, the layer which is formed from a polymer composition including the polymer matrix material and the at least one scavenger for moisture, oxygen, or a combination thereof, may be applied as a coating or second layer using any suitable method known in the art.

These and other aspects, embodiments and advantages of the present invention will become immediately apparent to those of ordinary skill in the art upon review of the Detailed Description and Claims to follow.

### BRIEF DESCRIPTION OF THE DRAWINGS

FIG. 1 is a side view of one embodiment of a stent protector.

FIG. 2 is a side view of another embodiment of a stent protector.

FIG. 3 is a radial cross-section taken at section 3-3 in FIGS. 1 and 2.

FIG. 4 is a side view of an embodiment of a stent protector according to the invention.

FIGS. 5-7 are radial cross-sections of various embodiments of multi-layer stent protectors.

FIG. 8 is a radial cross-section of a stent protector having a gradient of scavenger in a polymer layer.

FIG. 9 is a partial side view showing the distal end of a catheter assembly with parts shown in hidden line illustrating one embodiment of a stent protector.

FIG. 10 is a radial cross section taken at section 10-10 in FIG. 9.

FIG. 11 is a partial side view showing the distal end of a catheter assembly with parts shown in hidden line illustrating one embodiment of a stent protector.

FIG. 12 is a radial cross section taken at section 12-12 in FIG. 11.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

All U.S. patents and applications and all other published documents mentioned anywhere in this application are incorporated herein by reference in their entirety. Any copending patent applications, mentioned anywhere in this application are also hereby expressly incorporated herein by reference in their entirety

For the purposes of this disclosure, like reference numerals in the figures shall refer to like features unless otherwise indicated.

The present invention relates generally to a stent protector including a scavenger for moisture, oxygen, UV radiation, or a combination thereof. As used herein, the term “scavenger for moisture” shall be used interchangeably with desiccant, drying agent, moisture absorber, etc., and is a substance that is hygroscopic or that absorbs water from the surrounding environment.

“Oxygen scavengers” as used herein may include those compounds capable of absorbing oxygen, as well as reactive materials that may consume oxygen through chemical reaction. Typically, those capable of absorbing oxygen are inorganic in nature, while those which consume oxygen through chemical reaction are typically organic reactive materials.

As used herein, the term “scavenger for UV radiation” shall include those compounds which are also referred to as “UV absorbers”, “UV inhibitors” and “UV or light stabilizers”. Such terms are typically employed depending on the mode of action of the compound. For example, UV absorbers may function by shielding the polymer from UV radiation while UV or light stabilizers act by scavenging radical intermediates formed as a result of the photo-oxidation process. One example of a UV absorber is titanium oxide while examples of UV include the hindered amines.

Such materials may be in the form of finely divided particulate materials having an average particle size of about 0.1 microns to about 10 microns that can be suitably homogeneously dispersed in a polymer matrix material.

Examples of suitable moisture scavengers include, but are not limited to, silica gel, anhydrous calcium sulfate (anhydrite), calcium sulfate dihydrate (gypsum), calcium oxide, montmorillonite clay, molecular sieves such as those including natural or synthetic zeolite, activated alumina, para-toluene sulfonyl isocyanate, molecular sieves, oxazolidine, etc.

Anhydrous calcium sulfate is available from GypsumSolutions.com, The Industrial Products Division of United States Gypsum Company in Chicago, Ill., one example of which is CA-5 having an average particle size of about 1.4 microns.

Any suitable oxygen scavenger may be employed herein. Both inorganic and organic oxygen scavengers are available. Examples of inorganic oxygen scavengers include, but are not limited to, sulfites such as potassium sulfite, bisulfites, etc.

Examples of organic oxygen scavengers include, but are not limited to unsaturated hydrocarbons disclosed in U.S. Pat. No. 5,211,875, the entire content of which is incorporated by reference herein., ascorbic acid and its derivatives (including its alkali metal salts, optical isomers and derivatives thereof), ascorbate compounds as disclosed in U.S. Pat. No. 5,075,362 which is incorporated by reference herein in its entirety, etc.

Examples of classes of UV absorbers include, but are not limited to, benzophenone, benzotriazole, hydroxyphenyl triazine, benzoxazinone, oxanilide, benzylidene malonate, quinazoline, etc. See for example U.S. Pat. No. 7,173,128, the entire content of which is incorporated by reference herein.

UV absorbers and stabilizers are available from Ciba in Tarrytown, N.Y. under the tradenames of TINUVIN® and CHIMASSORB®

The scavengers may be employed singly, or in combinations of scavengers in amounts of about 0.1 wt-% to about 50 wt-%, more suitably about 0.5 wt-% to about 20 wt-% and most suitably about 1 wt-% to about 10 wt-% of the polymer composition.

The present invention does not exclude the use of other polymer additives such as antioxidants, plasticizers, colorants such as dyes and pigments, etc. which are known to those of skill in the art.

The protector, which may also be referred to as a protective sheath or sleeve, is generally provided for covering a device on the distal end of a catheter system prior to use such as during packaging, shipping and storage. The protector can be added to any delivery system prior to use and is intended to protect a deliverable device, such as an intravascular stent, stent-graft, etc. from being dislodged prematurely from the catheter system or moved with respect thereto.

Furthermore, the protector also works to prevent physical damage or contamination due to the transfer of unwanted material, and to protect any coating from being damaged prior to deployment of the stent.

Turning now to the figures, FIG. 1, illustrates generally at 10 one embodiment of a stent protector according to the invention. Stent protector 10 in this embodiment is a simple tubular member formed with a polymer layer 4 including a polymer matrix material and a scavenger 6 for moisture, oxygen or combination thereof, dispersed therein.

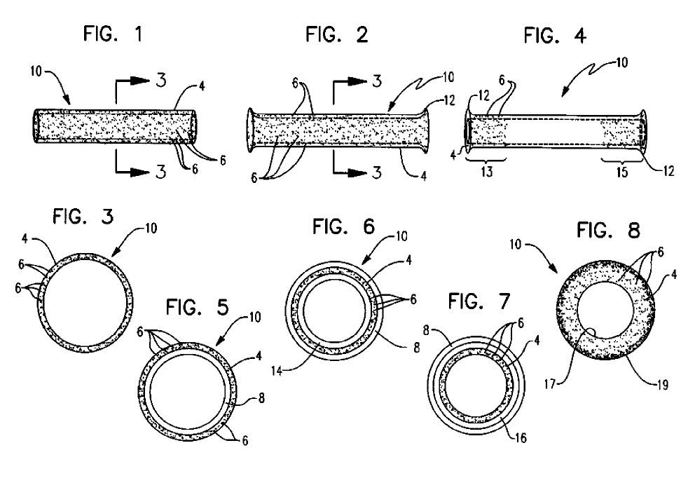

Stent protector 10 may have a flange 12 located at one of the proximal and/or distal ends, or both as shown in FIG. 2.

A radial cross section taken at section 3-3 in FIGS. 1 and 2 is shown in FIG. 3.

FIG. 4 is a side view of another embodiment of a stent protector 10 wherein layer 4 of stent protector 10 includes scavenger 6 for moisture, oxygen or UV radiation at the proximal end 13 and distal end 15 of the stent protector only. Other alternatives are contemplated. For example, layer could have scavenger in the central portion of the stent protector, or regions of the layer having the scavenger could run in a longitudinal direction rather than a radial direction as shown in FIG. 4. A similar layer 4 shown in FIG. 4 may also be employed in a multi-layer design such as shown in FIGS. 5-7 discussed in the following paragraphs.

Other embodiments wherein the stent protector is formed with two or more layers are shown as radial cross-sections in FIGS. 5-7. In one multi-layer embodiment shown in FIG. 5, stent protector 10 is formed with dual layers and includes an inner layer 8 and an outer layer 4. Outer layer 4 includes a scavenger 6 for moisture, oxygen, UV protection, or any combination thereof. Inner layer 8 may be formed from any suitable polymer composition and may include the same or a different polymer material than that of layer 4. In this embodiment, inner layer 8 does not include a scavenger for oxygen, moisture, UV radiation, or combination thereof.

Another multi-layer embodiment is shown in FIG. 6 wherein stent protector 10 is formed with three layers. An inner layer 14 may be formed from a water or moisture-permeable polymer composition, for example, various cellulose polymer materials such as methyl- and ethylcellulose and cellulose esters such as cellulose acetate, polyvinyl alcohol, polyurethanes which are permeable to water in vapor form including polyurethanes formed with ethylene oxide, etc. Microporous polymer films may also be employed. For example, porous fluoropolymers or polyolefins such as porous polytetrafluoroethylene (PTFE), also referred to as expanded PTFE available under the tradename of Gore-Tex®. The latter may be desirable due to its lubricious characteristics as well, thereby providing a surface that comes into contact with the stent which has a lower coefficient of friction These polymers are intended for illustrative purposes only, and not as a limitation on the scope of the present invention. Other water-permeable or semi-permeable polymer materials are known to those of skill in the art.

In any of the embodiments wherein multiple layers are employed, one of the layers may be formed so as to block UV radiation. For example, an outer layer may be highly filled and opaque so as to block UV radiation, or may be a composite layer such as a composite layer including a foil layer such as a metallized Mylar layer, for example.

Stent protector 10 shown in FIG. 6 further includes an intermediate layer 4 which includes the scavenger 6 for moisture, oxygen or combination thereof, and an outer layer 8 which may be formed from any suitable polymer composition.

Another embodiment of a multi-layer stent protector is illustrated in FIG. 7. In this embodiment, an inner layer 4 includes a scavenger 6 for moisture, while an intermediate layer 16 includes an oxygen scavenger. Outer layer 8 may be any suitable polymer composition.

FIG. 8 is a radial cross-section illustrating another embodiment wherein layer 4 of stent protector 10 has a gradient of scavenger 6 wherein the concentration of scavenger 6 increases from the inner surface 17 to the outer surface 19 of layer 4. A layer 4 as shown in FIG. 8 may be employed in a multilayer design such as is shown in FIGS. 5-7 as well.

The stent protector according to the invention may also have multi-layer construction at only certain portions of the stent protector, such as a polymer layer with the scavenger dispersed therein at the distal and/or proximal ends, or in the middle portion only. Furthermore, the multilayer layer construction can be configured such that the polymer layer with the scavenger has a gradient of thickness from one end of the stent protector to another.

The above examples are intended for illustrative purposes only, and not as a limitation on the scope of the present invention.

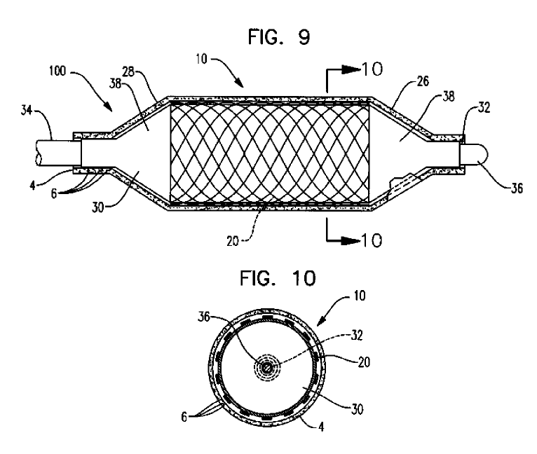

The stent protectors according to the invention may be used in combination with self-expanding or with balloon expandable stents. FIG. 9 illustrates one embodiment of a stent protector design which is shown in combination with a balloon-expandable stent. Stent protector 10 may be disposed about stent 20 which is disposed about balloon 30 at the distal end of a catheter assembly 100. As can be seen from the figure, in this embodiment catheter assembly 100 includes an inner shaft 32, an outer shaft 34, and a tip 36 disposed at the distal end of inner shaft 32. In this embodiment, the stent protector is configured and arranged such that it has tapered sections 28 which correspond to balloon waist portions 38. FIG. 10 is a radial cross section taken at section 10-10 in FIG. 9.

A similar design can be found in commonly assigned copending U.S. Patent Application Attorney Docket No. S63.2-13315US01, the entire content of which is incorporated by reference herein. Such stent protectors may be formed from heat shrink materials and may incorporate perforations so that it is readily removable from the catheter assembly as described therein.

In this embodiment, stent protector 10 is shown formed with a single layer 4 having a scavenger 6 for moisture, oxygen or a combination thereof disposed therein. Stent protector 10 may be formed with a multi-layer construction as well, examples of which are shown in FIGS. 5-7.

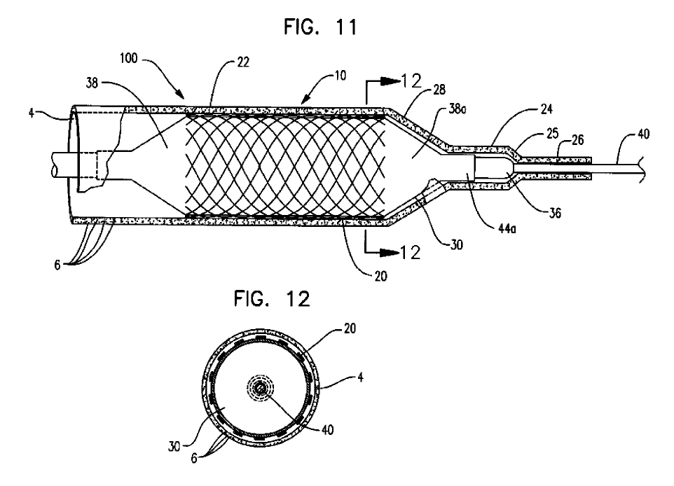

Another embodiment of a stent protector 10 is shown in combination with a balloon expandable stent 20 in FIG. 11. Stent protector 10 is shown disposed about stent 20 which is disposed about balloon 30 at the distal end of a catheter assembly 100. In this embodiment, stent protector 10 is designed with a protective portion 22 which fits loosely over stent 20 and a first tapered portion 28 which fits over distal balloon waist 38a, a first engagement region 24 which fits over balloon distal cone 44a, a second tapered region 25 configured to fit over tip 36 and a second engagement region 26 which engages product mandrel 40 which is removed prior to use of the catheter assembly in a procedure. In this embodiment, stent protector 10 is removed concurrently with product mandrel 40. A similar design may be found in commonly assigned copending U.S. Patent Application Attorney Docket No. S63.2-13457US01, the entire content of which is incorporated by reference herein.

This embodiment is shown also as a radial cross-section in FIG. 12 which is taken at section 12-12 in FIG. 11. Again, stent protector 10 is shown formed with a single polymer layer 4 having scavenger 6 for moisture, oxygen or a combination thereof dispersed therein. Again, multi-layer stent protectors may be employed as well, non-limiting examples of which are shown in FIGS. 5-7.

Another suitable stent protector design in which there is a stent covering portion which does not substantially engage the stent and an engagement portion is found in commonly assigned U.S. patent application Ser. No. 11/545/253 filed Oct. 10, 2006, the entire content of which is incorporated by reference herein.

The stent protectors described herein may also have inner diameters which are uniform throughout, or may have inner diameters which taper at one or both of the proximal and distal end such that the inner diameter is larger at one or both of the proximal and distal end.

Other examples of suitable stent protector designs can be found in commonly assigned U.S. Pat. Nos. 6,991,639, 6,416,529, 6,152,944, 6,132,450 and 5,893,868, each of which is incorporated by reference herein in its entirety. These examples are intended to be illustrative and not exhaustive, and do not limit the scope of the present invention.

The stent protector may be formed from any suitable material. Examples include, but are not limited to, fluoropolymers such as polytetrafluoroethylene (PTFE) and polyolefins such as polyethylene, for example, low density polyethylene (LDPE). Other suitable materials are generally known in the art and include, but are not limited to, polyamides, i.e. nylon, polyether block amides (PEBAX), polyesters such as polyethylene terephthalate (PET), silicone, POC and the like. In addition, the balloon and stent protectors of the present invention could be made of copolyesters such as Arnitel® EM 740 sold by DSM Engineering Plastics, as set forth in U.S. Pat. No. 5,556,383, incorporated herein by reference.

Heat shrinkable materials are typically thermoplastic, although in some instances thermoset materials may be employed, and include both elastomeric and non-elastomeric polymer materials. Suitable examples include, but are not limited to, polyolefins including, for example, homopolymers, copolymers and terpolymers of ethylene and propylene, fluoropolymers such as fluorinated ethylene-propylene (FEP), polytetrafluoroethylene (PTFE), polyvinylidene fluorides (PVFD) such as Kynar® PVFD's including Kynar® 500 available from Arkema Inc. in Philadelphia, Pa., copolymers of hexafluoropropylene (HFP), terpolymers of tetrafluoroethylene (TFE), ethylene-chlorotrifluoroethylene (ECTFE), VDF and HFP as well as perfluoromethylvinylether (PMVE), Viton® fluoropolymer elastomers available from Du Pont Performance Elastomers in Wilmington, Del., polyvinyl chloride (PVC), neoprene, silicon elastomers, polyamides including the nylons, polyether-block-amides, etc.

The above lists are intended for illustrative purposes only, and do not limit the scope of the present invention.

The scavenger may be mixed with a polymer material and the stent protector formed using any suitable method known in the art, examples of which including molding and extrusion. For stent protectors having tapered regions, crimping of a tubular member may also be employed. For multilayer structures, coextrusion or multilayer extrusion may be employed, or a tubular member may first be formed followed by coating. Any suitable coating method may be employed as well. Examples include, dipping, spraying, brushing, etc.

The present invention finds particular utility wherein stent coatings are employed. Stent coatings may incorporate a polymer material. There are any suitable polymer materials which are employed in stent coatings are such polymer materials are well known in the art.

In some embodiments, the stent coating may include bioresorbable polymers. Examples of bioresorbable polymers include, but are not limited to, polyhydroxyalkanoates such as poly(hydroxybutyrate) (PHB), poly(hydroxyvalerate) (PHV) and poly(hydroxybutyrate-co-valerate), polylactones such as polycaprolactone (PCL), poly(L-lactic acid) (PLA), poly(glycolic acid), poly(D,L-lactic acid), poly(lactide-co-glycolide) (PLGA), polydioxanone, polyorthoesters, polyanhydrides, poly(glycolic acid-co-trimethylene carbonate), polyphosphoesters, polyphosphoester urethanes, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen, hyaluronic acid, etc., and mixtures thereof.

Other stent coatings may be formed of a sintered metal, for example.

Lubricious coatings are also commonly employed on various components of a catheter assembly and are also well known in the art. Lubricious coatings include both hydrophilic and non-hydrophilic polymer materials. Commonly employed hydrophilic polymer materials include those referred to in the art as hydrogels.

Lubricious coatings may be employed on any component of the catheter assembly and are commonly employed on the balloon body, waist and cones, or any combination thereof, as well as on the outer catheter shaft and the catheter distal tip.

Any of the coatings may incorporate a therapeutic agent therein. The terms, “therapeutic agent”, “drug”, “pharmaceutically active agent”, “pharmaceutically active material”, “beneficial agent”, “bioactive agent”, and other related terms may be used interchangeably herein and include genetic therapeutic agents, non-genetic therapeutic agents and cells. A drug may be used singly or in combination with other drugs. Drugs include genetic materials, non-genetic materials, and cells.

Examples of drugs can be found in commonly assigned U.S. Pat. Nos. 7,105,175, 7,014,654, 6,899,731, 6,855,770 and 6,545,097, each of which is incorporated by reference herein in its entirety, and in commonly assigned U.S. Patent Application Publication No. 2004/0215169, the entire content of which is incorporated by reference herein.

The above disclosure is intended to be illustrative and not exhaustive. This description will suggest many variations and alternatives to one of ordinary skill in this art. Those familiar with the art may recognize other equivalents to the specific embodiments described herein which equivalents are also intended to be encompassed by the claims.

1. A protector for an intraluminal medical device, the protector comprising a tubular member, said tubular member having a layer, said layer comprising a polymer composition, the polymer composition comprising a polymer matrix material and at least one scavenger for moisture, oxygen, UV radiation or a combination thereof dispersed in said polymer matrix material.

2. The protector of claim 1 comprising at least one second layer.

3. The protector of claim 1 wherein said at least one scavenger is a moisture scavenger.

4. The protector of claim 3 wherein said moisture scavenger comprises at least one member selected from the group consisting of calcium sulfate, molecular sieves, montmorillonite clay, activated alumina and mixtures thereof.

5. The protector of claim 3 wherein said moisture scavenger comprises anhydrous calcium sulfate, calcium sulfate dihydrate or a mixture thereof.

6. The protector of claim 1 wherein said tubular member comprises a proximal end and a distal end, said tubular member comprising a flange at said proximal end, said distal end or both.

7. The protector of claim 1 wherein said tubular member comprises at least one taper region at said distal end of said protector.

8. The protector of claim 1 wherein said polymer matrix material is about 0.1 to about 20 percent by weight of said polymer composition.

9. The protector of claim 1 wherein said polymer composition comprises a moisture scavenger in an amount of about 0.2 to about 10 percent by weight of said polymer composition.

10. The protector of claim 1 disposed about a stent.

11. The protector of claim 1 disposed about a balloon.

12. The protector of claim 10 wherein said stent comprises a drug eluting coating.

13. The protector of claim 12 wherein said drug eluting coating comprises at least one polymer or at least one therapeutic agent which is susceptible to hydrolysis in the presence of moisture.

14. A catheter assembly comprising:
at least one catheter shaft having a distal end and a proximal end;
a medical device disposed about the distal end of the catheter; and
a protector disposed about the medical, the protector comprising a tubular member formed from a polymer composition, the polymer composition comprising a polymer matrix material and at least one scavenger for moisture,
oxygen, UV radiation or a combination thereof dispersed in said polymer matrix material.

15. The catheter assembly of claim 14 wherein said medical device is a stent.

16. The catheter assembly of claim 15 wherein said stent is disposed about an expandable medical balloon.

17. The catheter assembly of claim 14 wherein said at least one scavenger is a moisture scavenger.

18. The catheter assembly of claim 14 wherein said moisture scavenger comprises at least one member selected from the group consisting of calcium sulfate, molecular sieves and mixtures thereof.

19. The catheter assembly of claim 14 wherein said at least one moisture scavenger is anhydrous calcium sulfate, calcium sulfate dihydrate or a mixture thereof.

20. The catheter assembly of claim 15 wherein said stent comprises a drug eluting coating.

21. The catheter assembly of claim 20 wherein said drug eluting coating comprises at least one polymer that is susceptible to hydrolysis in the presence of moisture.

22. The catheter assembly of claim 21 wherein said at least one polymer is a member selected from the group consisting of homopolymers and copolymers of l-lactic acid, D,L-lactic acid, glycolic acid and mixtures thereof.

23. A method of making a protector for a medical device, the method comprising:
mixing at least one polymer material with at least one scavenger for moisture, oxygen, UV radiation or a combination thereof; and
forming a tubular member.

24. A method of protecting a stent from moisture, oxygen or radiation, the method comprising:
providing a protector, the protector comprising at least one polymer layer, the polymer layer comprising a scavenger for moisture, oxygen, radiation, or a combination thereof; and
disposing said protector about said stent.
